(19) United States
(12) Patent Application Publication
(10) Pub. No.: US 2026/0272462 A1
(43) Pub. Date: Sep. 17, 2026

(54) Handheld Electromechanical Surgical System With Low Tissue Compression Indiction

(71) Applicant: Covidien LP, Mansfield, MA (US)

(72) Inventors: Steven H. JOYCE, North Haven, CT (US); Drew R. SEILS, North Haven, CT (US); Alexander J. HART, North Haven, CT (US); Stephen R. PAUL, North Haven, CT (US)

(73) Assignee: Covidien LP, Mansfield, MA (US)

(21) Appl. No.: 19/099,111

(22) PCT Filed: Jul. 27, 2023
(86) PCT No.: PCT/US2023/071075

§ 371 Date: Jan. 28, 2025

Related U.S. Application Data
(60) Provisional application No. 63/393,326, filed on Jul. 29, 2022.

(51) Int. Cl. A61B 17/115; A61B 17/00; A61B 90/00

(52) U.S. Cl. CPC A61B 17/1155; A61B 90/06; A61B 2017/00119; A61B 2017/00398; A61B 2017/00473; A61B 2090/064

(57) ABSTRACT

A surgical device includes a reload assembly having a plurality of staples and a storage device storing data including a minimum clamp force limit. The surgical device also includes an anvil assembly movable relative to the reload assembly, a power source, and a motor coupled to the power source. The surgical device further includes a transmission assembly movable by the motor and configured to move the anvil assembly relative to the reload assembly. The surgical device also includes a force sensor configured to measure a force imparted on the anvil assembly by the transmission assembly. The surgical device also includes a controller configured to activate the motor to move the anvil assembly relative to the reload assembly, compare the measured force to the minimum clamp force limit, and output an alert in response to the measured force being lower than the minimum clamp force limit.

### CROSS-REFERENCE TO RELATED APPLICATIONS

This application claims the benefit of the filing date of provisional U.S. Patent Application No. 63/393,326 filed on Jul. 29, 2022. The entire disclosure of the foregoing application is incorporated by reference herein.

### BACKGROUND

### 1. Technical Field

The present disclosure relates to surgical devices. More specifically, the present disclosure relates to handheld electromechanical surgical systems for performing surgical procedures.

### 2. Background of Related Art

Circular staplers are used in a surgical procedure to reattach rectum portions that were previously transected, or similar procedures. Circular clamping, cutting and stapling instruments may be manually actuated and may include a pistol or linear grip-styled structure having an elongated shaft extending therefrom and a staple cartridge supported on the distal end of the elongated shaft. A physician may insert an anvil assembly of the circular stapling instrument through an incision and toward the transected rectum portions. The physician may also insert the remainder of the circular stapling instrument (including the cartridge assembly) into a rectum of a patient and maneuver the instrument up the colonic tract of the patient toward the transected rectum portions. The anvil and cartridge assemblies are approximated toward one another, and staples are ejected from the cartridge assembly toward the anvil assembly to form the staples in tissue to affect an end-to-end anastomosis, and an annular knife is advanced to core a portion of the clamped tissue portions. After the end-to-end anastomosis has been affected, the circular stapling apparatus is removed from the surgical site. Powered surgical staplers have also been developed and utilize one or more motors to clamp, cut, and staple tissue. Due to variations in patient anatomy, the thickness of tissue being stapled varies considerably. Thus, there is a need to determine tissue thickness during stapling procedures.

### SUMMARY

The present disclosure provides a powered circular stapler that is configured to operate in four sequences, namely, clamping, stapling, cutting, and unclamping to form an anastomosis by connecting two portions of a structure (e.g., intestine, colon, etc.). The powered circular stapler includes a handle assembly having a power source and one or more motors coupled to the power source. The stapler also includes an adapter assembly having multiple transmission assemblies, e.g., drive shafts, which transmit actuation from the powered handle. The powered handle assembly and the adapter assembly may be reusable.

The powered surgical staplers operate in four phases, namely, clamping, stapling, cutting, and unclamping. Clamping is accomplished by moving the anvil in a proximal direction to compress tissue between the anvil and a reload assembly, which includes a plurality of staples. The anvil and the reload assembly may be disposable. During stapling, the staples are ejected from the reload assembly into the clamped tissue and are deformed against the anvil. Cutting includes moving an annular knife through the compressed and stapled tissue until the knife contacts the anvil. During unclamping, the anvil assembly is moved distally away from the cut tissue and the reload assembly.

Each of the clamping, stapling, cutting, and unclamping phases may be monitored to ensure proper operation of the powered surgical stapler, including proper anvil release after the cutting process is complete. The powered surgical stapler includes a sensor, e.g., a strain gauge, configured to measure forces during each of the processes, and a controller configured to monitor the forces and detect any abnormalities during operation of the powered surgical stapler. The powered stapler is configured to operate with a plurality of different sized reloads, which may have a diameter from about 20 mm to about 35 mm and correspondingly sized anvils. The minimum clamp force may be stored in the device software that resides on the handle or any other suitable location, such as a cloud service. In embodiments, each of the reloads may include a storage device storing a variety of parameters including minimum clamp force limit.

The powered surgical stapler is configured to monitor clamp forces during the surgery and to determine tissue thickness being compressed between the anvil and the reload. This allows the user to select a more appropriate end effector, e.g., downsize, that fits a lower thickness indication to ensure a better outcome and less potential for leaks. Thus, this disclosure provides a method for determining if the tissue compression pressure is lower than recommended for the chosen end effector and conveying that information to the surgeon prior to firing. This allows the surgeon to make a more informed decision based on immediate feedback from the device.

The method includes establishing a minimum clamp force limit for each size end effector such that the equivalent “low pressure limit” is the same for all sizes of end effectors, since pressure is calculated by dividing applied force by the surface area of the end effector. Since the surface area that comes in contact with the tissue varies for the different end effectors, a different “minimum clamp force limit” is established for each end effector, such that a “low tissue pressure limit” is the same across all sizes. So when clamping to the specified tissue gap for that particular end effector, if the final clamp force at the specified tissue gap is below the minimum clamp force limit, the powered surgical stapler displays or provides some other indication to the user that the tissue pressure may be too low and alerting them that they may want to assess the situation and make an informed decision as to whether to fire, downsize, or create the anastomosis in some other manner (e.g., sutures, etc.).

According to one embodiment of the present disclosure, a surgical device is disclosed. The surgical device includes a reload assembly having a plurality of staples. The surgical device also includes an anvil assembly movable relative to the reload assembly, a power source, and a motor coupled to the power source. The surgical device further includes a transmission assembly movable by the motor and configured to move the anvil assembly relative to the reload assembly. The surgical device also includes a force sensor configured to measure a force imparted on the anvil assembly by the transmission assembly and a storage device storing data including a minimum clamp force limit. The surgical device also includes a controller configured to activate the motor to move the anvil assembly relative to the reload assembly, compare the measured force to the minimum clamp force limit, and output an alert in response to the measured force being lower than the minimum clamp force limit.

Implementations of the above embodiment may include one or more of the following features. According to one aspect of the above embodiment, the data may further include a clamping distance. The controller may be further configured to activate the motor to move the anvil assembly relative to the reload assembly until the clamp distance is reached. The controller may be also configured to compare the force measured at the clamping distance to the minimum clamp force limit. The controller may be further configured to enable ejection of the plurality of staples in response to the measured force being higher than the minimum clamp force limit. The controller may be also configured to determine an alternative reload assembly in response to the measured force being lower than the minimum clamp force limit. The surgical device may also include a display screen configured to display at least one of the alert or information pertaining the alternative reload.

According to another embodiment of the present disclosure, a method for controlling a surgical device is disclosed. The method includes receiving, at a controller, minimum clamp force limit stored in a storage device. The method also includes activating a motor to move a transmission assembly coupled to an anvil assembly that is movable relative to a reload assembly having a plurality of staples. The method further includes measuring, through a force sensor, force imparted on the anvil assembly by the transmission assembly. The method also includes comparing, at the controller, the measured force to the minimum clamp force limit. The method further includes outputting, by the controller, an alert in response to the measured force being lower than the minimum clamp force limit.

Implementations of the above embodiment may include one or more of the following features. According to one aspect of the above embodiment, the method may further include receiving, at the controller, a clamping distance stored in the storage device of the reload. The method may also include activating the motor to move the anvil assembly relative to the reload assembly until the clamp distance is reached. The method may additionally include comparing, at the controller, the force measured at the clamping distance to the minimum clamp force limit. The method may further include enabling ejection of the plurality of staples in response to the measured force being higher than the minimum clamp force limit. The method may also include determining, at the controller, an alternative reload assembly in response to the measured force being lower than the minimum clamp force limit. The method may further include outputting on a display screen at least one of the alert or information pertaining to the alternative reload.

According to a further embodiment of the present disclosure, a surgical device is disclosed. The surgical device includes a reload assembly having a plurality of staples and a storage device storing data pertaining to the reload assembly. The data includes minimum clamp force limit and clamping distance. The device also includes an anvil assembly movable relative to the reload assembly. The device further includes a power source and a motor coupled to the power source. The device further includes a transmission assembly movable by the motor and configured to move the anvil assembly relative to the reload assembly. The device additionally includes a force sensor configured to measure force imparted on the anvil assembly by the transmission assembly. The device further includes a display and a controller configured to: activate the motor to move the anvil assembly relative to the reload assembly until the clamping distance is reached; compare the force measured at the clamping distance to the minimum clamp force limit; and output an alert on the display in response to the measured force being lower than the minimum clamp force limit.

Implementations of the above embodiment may include one or more of the following features.

In another embodiment, if the clamp force is below the minimum clamp force limit when the anvil reaches the normal clamping distance, the stapler may continue to move the anvil in the proximal direction towards the reload, going below the normal clamping distance, and then stop clamping when the minimum clamping force is achieved. At this point the stapler displays or provides some other indication to the user that they may want to assess the situation and make an informed decision as to whether to fire, downsize, or create the anastomosis in some other manner (e.g., sutures, etc.). If they chose to fire in this reduced clamp gap condition, the device will adjust the staple and cut stroke to compensate for the more proximal position of the anvil to ensure good staple formation and cutting of the anastomosis in this reduced gap state, which is less than normal clamp gap.

### BRIEF DESCRIPTION OF THE DRAWINGS

Embodiments of the present disclosure are described herein with reference to the accompanying drawings, wherein:

FIG. 1 is a perspective view of a powered circular stapler including a handle assembly, an adapter assembly, and an end effector, according to an embodiment of the present disclosure;

FIG. 2 is a schematic diagram of the handle assembly, the adapter assembly, and the end effector of FIG. 1;

FIG. 3 is a side perspective view of the adapter assembly and the end effector, an annular reload and an anvil assembly, attached to the adapter assembly of FIG. 1 according to an embodiment of the present disclosure;

FIG. 4 is a perspective view of a clamping transmission assembly disposed within the adapter assembly of FIG. 1, shown partially in phantom;

FIG. 5 is a perspective view of a stapling transmission assembly disposed within the adapter assembly of FIG. 1, shown partially in phantom;

FIG. 6 is a perspective view of a cutting transmission assembly disposed within the adapter assembly of FIG. 1, shown partially in phantom;

FIG. 7 is a cross-sectional view of a reload of the end effector of FIG. 1;

FIG. 8 is a perspective view of the adapter assembly, shown partially disassembled, with a strain gauge assembly; and

FIGS. 9A and 9B is a flow chart of a method for determining low tissue compression indication according to an embodiment of the present disclosure.

### DETAILED DESCRIPTION OF EMBODIMENTS

Embodiments of the presently disclosed surgical devices, and adapter assemblies for surgical devices and/or handle assemblies are described in detail with reference to the drawings, in which like reference numerals designate identical or corresponding elements in each of the several views. As used herein the term “distal” refers to that portion of the surgical instrument, or component thereof, farther from the user, while the term “proximal” refers to that portion of the surgical instrument, or component thereof, closer to the user.

The present disclosure provides a powered circular stapler 10 having a handle assembly, an adapter assembly coupled to the handle assembly, and an end effector coupled to the adapter assembly. The stapler allows for full, independent control of four functions: clamping, stapling, cutting, and unclamping. This allows certain portions of the stapler to adapt if the tissue presents a non-ideal situation.

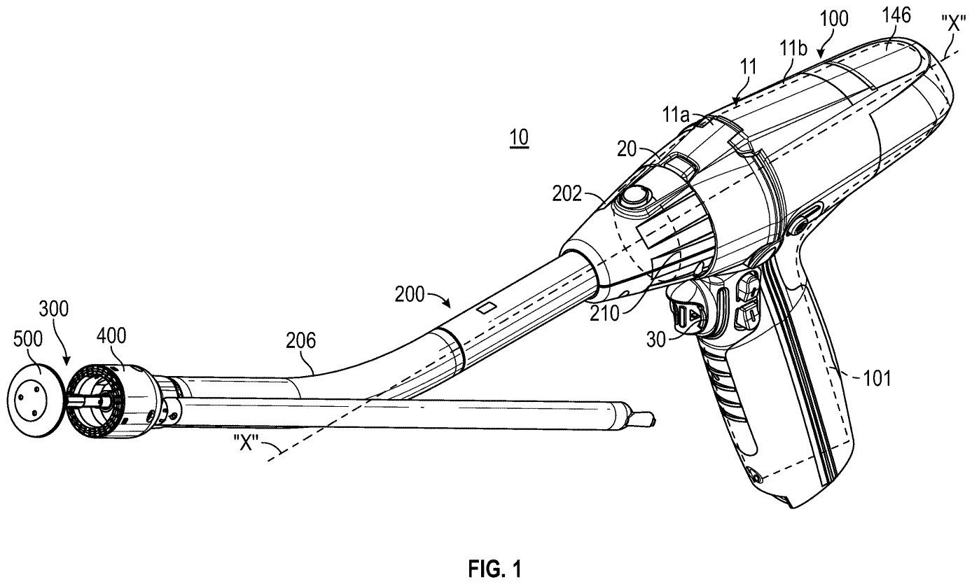

FIG. 1 illustrates a surgical device, such as, for example, a powered circular stapler 10 for forming end-to-end anastomosis (“EEA”), including a handle assembly 100, which is configured for selective connection with an adapter assembly 200. The adapter assembly 200 is configured for selective connection with an end effector 300, which includes a reload 400 and an anvil assembly 500. The end effector 300 is configured to produce a surgical effect on tissue of a patient, namely, forming an anastomosis by connecting two portions of a structure (e.g., intestine, colon, etc.) by clamping, stapling, and cutting tissue grasped within the end effector 300.

The handle assembly 100 includes a power handle 101 and an outer shell housing 11 configured to selectively receive and encase power handle 101. The shell housing 11 includes a distal half-section 11a and a proximal half-section 11b pivotably connected to distal half-section 11a. When joined, distal and proximal half-sections 11a, 11b define a shell cavity therein in which power handle 101 is disposed.

While the powered circular stapler 10 is described herein as a modular device including a plurality of interconnected components, such as the handle assembly 100, the removable shell housing 11, and the adapter assembly 200, etc., the powered circular stapler 10 may be formed as an integrated device with one or more of the components being securely attached to each other, e.g., during manufacturing of the powered circular stapler.

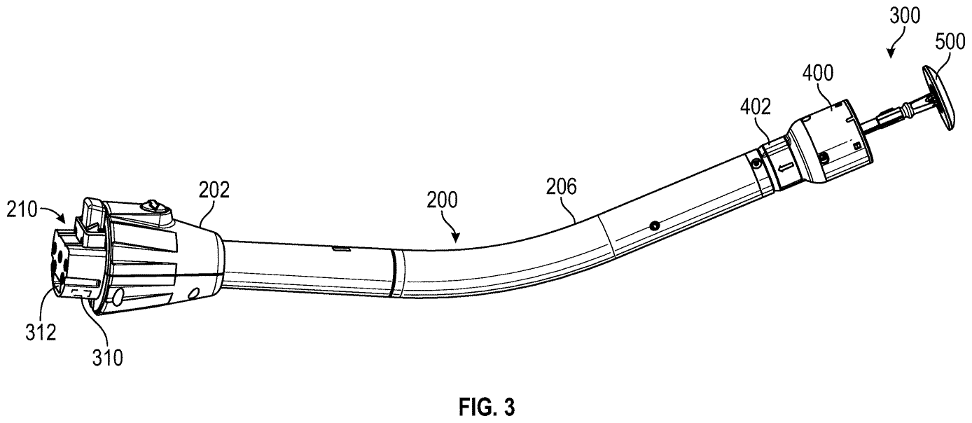

Distal and proximal half-sections 11a, 11b of shell housing 11 are divided along a plane that traverses a longitudinal axis “X” of adapter assembly 200. Distal half-section 11a of shell housing 11 defines a connecting portion 20 configured to accept a corresponding drive coupling assembly 210 (FIG. 3) of adapter assembly 200. Distal half-section 11a of shell housing 11 supports a toggle control button 30. Toggle control button 30 is capable of being actuated in four directions (e.g., a left, right, up, and down).

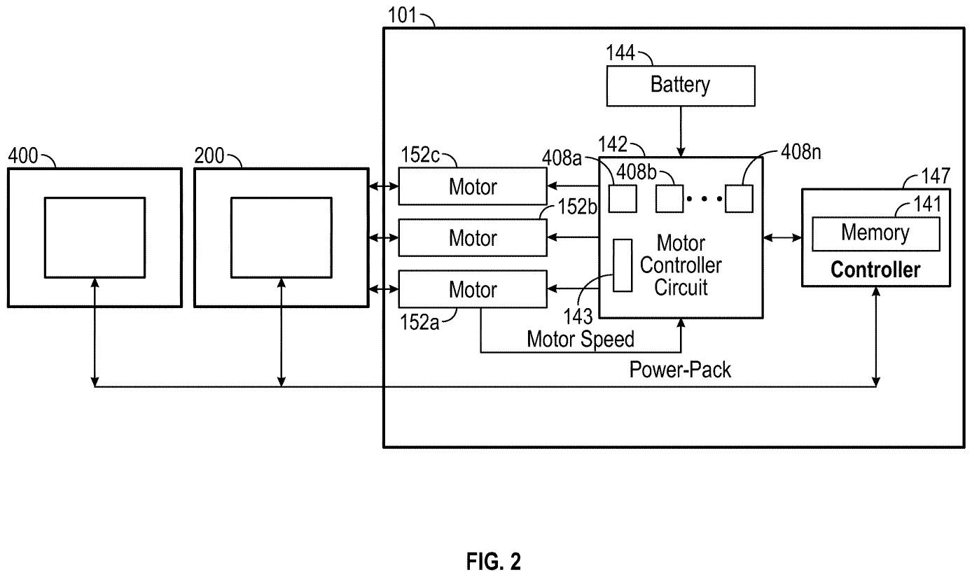

With reference to FIGS. 1 and 2, the power handle 101 includes a main controller circuit board 142, a rechargeable battery 144 configured to supply power to any of the electrical components of handle assembly 100, and a plurality of motors, i.e., a first motor 152a, a second motor 152b, a third motor 152c coupled to the battery 144. The power handle 101 also includes a display 146. In embodiments, the motors 152a, 152b, 152c may be coupled to any suitable power source configured to provide electrical energy to the motors 152a, 152b, 152c, such as an AC/DC transformer. Each of the motors 152a, 152b, 152c is coupled a motor controller 143 which controls the operation of the corresponding motors 152a, 152b, 152c including the flow of electrical energy from the battery 144 to the motors 152a, 152b, 152c. A main controller 147 is provided that controls the power handle 101. The main controller 147 is configured to execute software instructions embodying algorithms disclosed herein, such as clamping, stapling, and cutting algorithms which control operation of the power handle 101.

The motor controller 143 includes a plurality of sensors 408a . . . 408n configured to measure operational states of the motors 152a, 152b, 152c and the battery 144. The sensors 408a-n include a strain gauge 408b and may also include voltage sensors, current sensors, temperature sensors, telemetry sensors, optical sensors, and combinations thereof. The sensors 408a-408n may measure voltage, current, and other electrical properties of the electrical energy supplied by the battery 144. The sensors 408a-408n may also measure angular velocity (e.g., rotational speed) as revolutions per minute (RPM), torque, temperature, current draw, and other operational properties of the motors 152a, 152b, 152c. The sensor 408a also includes an encoder configured to count revolutions or other indicators of the motors 152a, 152b, 152c, which is then use by the main controller 147 to calculate linear movement of components movable by the motors 152a, 152b, 152c. Angular velocity may be determined by measuring the rotation of the motors 152a, 152b, 152c or a drive shaft (not shown) coupled thereto and rotatable by the motors 152a, 152b, 152c. The position of various axially movable drive shafts may also be determined by using various linear sensors disposed in or in proximity to the shafts or extrapolated from the RPM measurements. In embodiments, torque may be calculated based on the regulated current draw of the motors 152a, 152b, 152c at a constant RPM. In further embodiments, the motor controller 143 and/or the main controller 147 may measure time and process the above-described values as a function of time, including integration and/or differentiation, e.g., to determine the rate of change in the measured values. The main controller 147 is also configured to determine distance traveled of various components of the adapter assembly 200 and/or the end effector 300 by counting revolutions of the motors 152a, 152b, 152c.

The motor controller 143 is coupled to the main controller 147, which includes a plurality of inputs and outputs for interfacing with the motor controller 143. In particular, the main controller 147 receives measured sensor signals from the motor controller 143 regarding operational status of the motors 152a, 152b, 152c and the battery 144 and, in turn, outputs control signals to the motor controller 143 to control the operation of the motors 152a, 152b, 152c based on the sensor readings and specific algorithm instructions. The main controller 147 is also configured to accept a plurality of user inputs from a user interface (e.g., switches, buttons, touch screen, etc.) coupled to the main controller 147.

The main controller 147 is also coupled to a storage device 141, which may include volatile (e.g., RAM) and non-volatile storage configured to store data, including software instructions for operating the power handle 101. The main controller 147 is also coupled to the strain gauge 408b of the adapter assembly 200 using a wired or a wireless connection and is configured to receive strain measurements from the strain gauge 408b which are used during operation of the power handle 101.

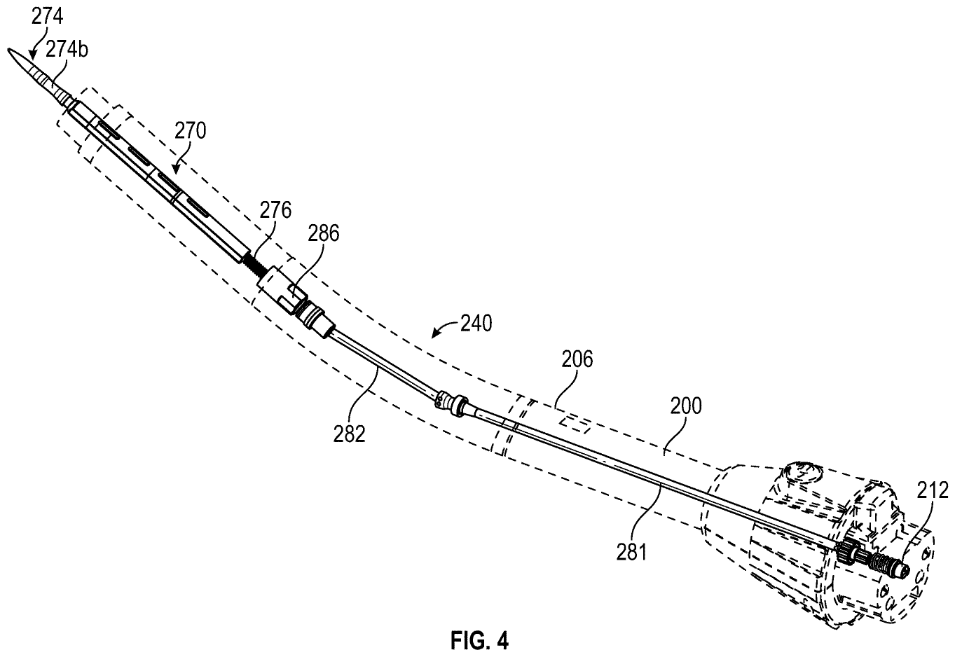

The power handle 101 includes a plurality of motors 152a, 152b, 152c each including a respective motor shaft (not explicitly shown) extending therefrom and configured to drive a respective transmission assembly. Rotation of the motor shafts by the respective motors function to drive shafts and/or gear components of adapter assembly 200 in order to perform the various operations of handle assembly 100. In particular, motors 152a, 152b, 152c of power handle 101 are configured to drive shafts and/or gear components of adapter assembly 200 in order to selectively extend/retract a trocar member 274 (FIG. 4) of a trocar assembly 270 of adapter assembly 200. Extension/retraction of the trocar member 274 opens/closes end effector 300 (when anvil assembly 500 is connected to trocar member 274 of trocar assembly 270), fire an annular array of staples 423 of reload 400, and move an annular knife 444 of reload 400.

Turning now to FIGS. 3 and 4, adapter assembly 200 includes an outer knob housing 202 and an outer tube 206 extending from a distal end of knob housing 202. Knob housing 202 and outer tube 206 are configured and dimensioned to house the components of adapter assembly 200. The knob housing 202 includes an electrical connector 312 and a storage device 310 coupled thereto. The storage device 310 is configured to store various operating parameters pertaining to the adapter assembly 200. Adapter assembly 200 is configured to convert rotation of coupling shafts (not explicitly shown) of handle assembly 100 into axial translations useful for operating trocar assembly 270 of adapter assembly 200, anvil assembly 500, and/or staple driver 430 or knife assembly 440 of reload 400.

Adapter assembly 200 further includes the trocar assembly 270 removably supported in a distal end of outer tube 206. Trocar assembly 270 includes a trocar member 274 and a drive screw 276 operably received within trocar member 274 for axially moving trocar member 274 relative to outer tube 206. A distal end 274b of trocar member 274 is configured to selectively engage anvil assembly 500, such that axial movement of trocar member 274, via a rotation of drive screw 276, results in a concomitant axial movement of anvil assembly 500.

With reference to FIG. 4, a clamping transmission assembly 240 includes first rotatable proximal drive shaft 212 coupled to the first motor 152a, a second rotatable proximal drive shaft 281, a rotatable distal drive shaft 282, and a coupling member 286, each of which are supported within the outer tube 206 of adapter assembly 200. Clamping transmission assembly 240 functions to extend/retract trocar member 274 of trocar assembly 270 of adapter assembly 200, and to open/close the anvil assembly 500 when anvil assembly 500 is connected to trocar member 274.

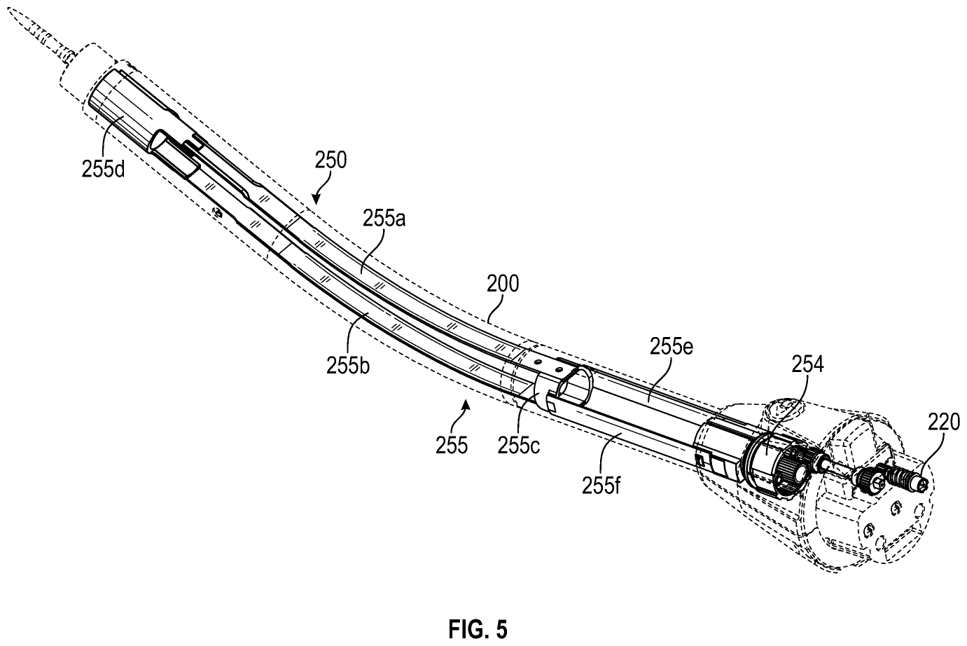

With reference to FIG. 5, the adapter assembly 200 includes a stapling transmission assembly 250 for interconnecting the second motor 152b and a second axially translatable drive member of reload 400, wherein the stapling transmission assembly 250 converts and transmits a rotation of the second motor 152b to an axial translation of an outer flexible band assembly 255 of adapter assembly 200, and in turn, the staple driver 430 of reload 400 to fire staples 423 from the reload 400 and against anvil assembly 500.

The stapling transmission assembly 250 of adapter assembly 200 includes the outer flexible band assembly 255 secured to staple driver coupler 254. A second rotatable proximal drive shaft 220 is coupled to the second motor 152b and is configured to actuate that staple driver coupler 254, which converts rotational movement into longitudinal movement. Outer flexible band assembly 255 includes first and second flexible bands 255a, 255b laterally spaced and connected at proximal ends thereof to a support ring 255c and at distal ends thereof to a proximal end of a distal pusher 255d. Each of first and second flexible bands 255a, 255b is attached to support ring 255c and distal pusher 255d. Outer flexible band assembly 255 further includes first and second connection extensions 255e, 255f extending proximally from support ring 255c. First and second connection extensions 255e, 255f are configured to operably connect outer flexible band assembly 255 to staple driver coupler 254 of stapling transmission assembly 250.

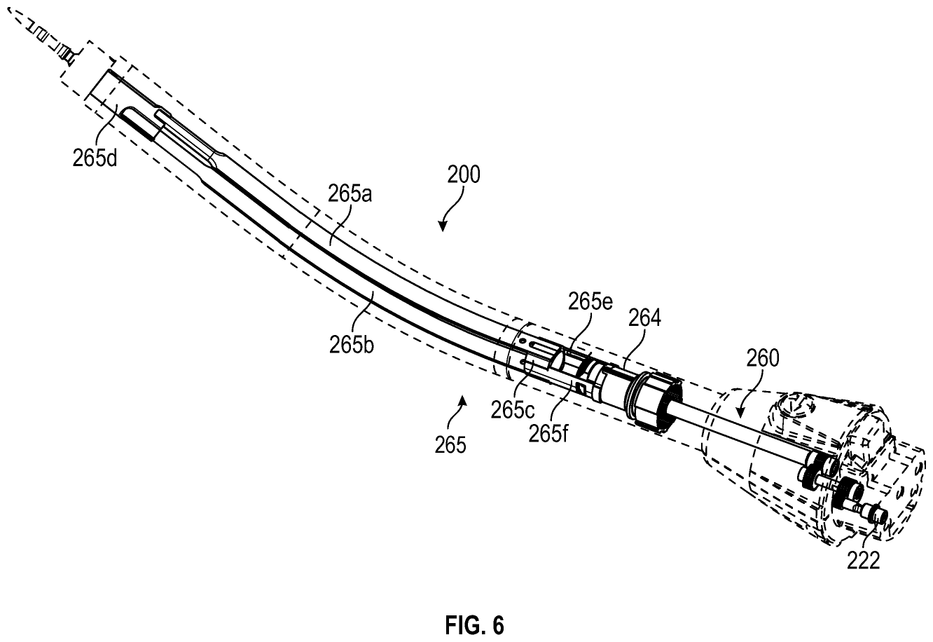

With reference to FIG. 6, the adapter assembly 200 also includes a cutting transmission assembly 260 having a third rotatable proximal drive shaft 222 for interconnecting the third motor 152c and the annular knife 444 of reload 400, wherein the cutting transmission assembly 260 converts and transmits a rotation of one of the third motor 152c to an axial translation of an inner flexible band assembly 265 of adapter assembly 200, and in turn, a knife carrier 442 of reload 400 to advance the annular knife 444 from the reload 400 and against anvil assembly 500.

Inner flexible band assembly 265 includes first and second flexible bands 265a, 265b laterally spaced and connected at proximal ends thereof to a support ring 265c and at distal ends thereof to a proximal end of a support base 265d. Each of first and second flexible bands 265a, 265b are attached to support ring 265c and support base 265d.

Inner flexible band assembly 265 further includes first and second connection extensions 265e, 265f extending proximally from support ring 265c. First and second connection extensions 265e, 265f are configured to operably connect inner flexible band assembly 265 to knife driver 264 of cutting transmission assembly 260. Support base 265d extends distally from flexible bands 265a, 265b and is configured to connect with a knife assembly 440 of reload 400.

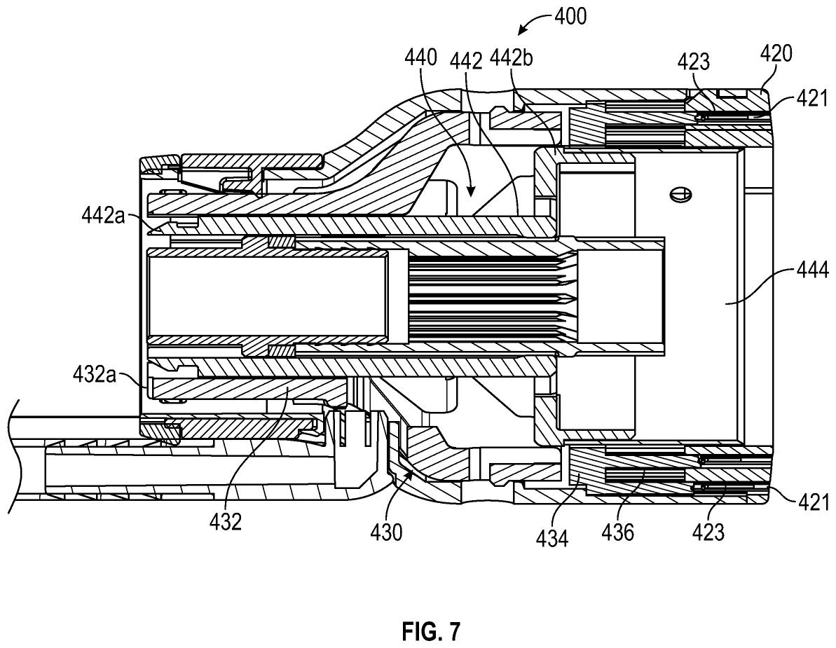

With reference to FIG. 7, staple driver 430 of reload 400 includes a staple cartridge 420 having a driver adapter 432 and a driver 434. A proximal end 432a of driver adapter 432 is configured for selective contact and abutment with distal pusher 255d of outer flexible band assembly 255 of stapling transmission assembly 250 of adapter assembly 200. In operation, during distal advancement of outer flexible band assembly 255, as described above, distal pusher 255d of outer flexible band assembly 255 contacts proximal end 432a of driver adapter 432 to advance driver adapter 432 and driver 434 from a first or proximal position to a second or distal position. Driver 434 includes a plurality of driver members 436 aligned with staple pockets 421 of staple cartridge 420 for contact with staples 423. Accordingly, advancement of driver 434 relative to staple cartridge 420 causes ejection of the staples 423 from staple cartridge 420.

The knife assembly 440 of the reload 400 includes a knife carrier 442 and an annular knife 444 secured about a distal end 442b of knife carrier 442. A proximal end 442a of knife carrier 442 is configured to engage the support base 265d of inner flexible band assembly. In operation, during distal advancement of inner flexible band assembly 265, support base 265d of inner flexible band assembly 265 connects with proximal end 442a of knife carrier 442 to advance knife carrier 442 and annular knife 444 from a first or proximal position to a second or advanced position to cause the cutting of tissue disposed between staple cartridge 420 and anvil assembly 500.

Forces during an actuation of trocar member 274, closing of end effector 300 (e.g., a retraction of anvil assembly 500 relative to reload 400), ejecting staples 423 from the reload 400, and advancement of the knife assembly 440 may be measured by the strain gauge 408b in order to monitor and control various processes, such as firing of staples 423 from reload 400; monitor forces during a firing and formation of the staples 423 as the staples 423 are being ejected from reload 400; optimize formation of the staples 423 (e.g., staple crimp height) as the staples 423 are being ejected from reload 400 for different indications of tissue; and monitor and control a firing of the annular knife of reload 400.

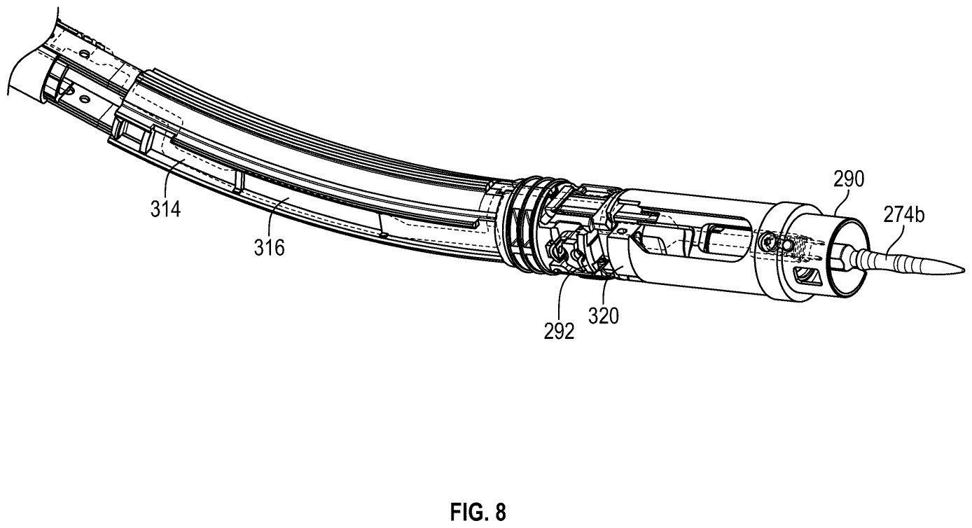

With reference to FIG. 8, the strain gauge 408b of adapter assembly 200 is disposed within a strain gauge housing 320. The strain gauge 408b measures and monitors the retraction of trocar member 274 as well as the ejection and formation of the staples 423 from the reload 400. During the closing of end effector 300, when anvil assembly 500 contacts tissue, an obstruction, a tissue-contacting surface of the reload 400, staple ejection, or the like, a reaction force is exerted on anvil assembly 500 which is in a generally distal direction. This distally directed reaction force is communicated from anvil assembly 500 to the strain gauge 408b. The strain gauge 408b then communicates signals to main controller circuit board 142 of power handle 101 of handle assembly 100. Graphics are then displayed on the display screen 146 of handle assembly 100 to provide the user with real-time information related to the status of the firing of handle assembly 100.

The trocar assembly 270 is axially and rotationally fixed within outer tube 206 of adapter assembly 200. With reference to FIG. 8, adapter assembly 200 includes a support block 292 fixedly disposed within outer tube 206. The strain gauge housing 320 is disposed between the support block 292 and a connector sleeve 290. The reload 400 is removably coupled to the connector sleeve 290.

In operation, strain gauge 408b of adapter assembly 200 measures and monitors the retraction of trocar member 274, which passes through the strain gauge 408b. The strain gauge 408b of adapter assembly 200 also measures and monitors ejection of the staples 423 from the reload 400, since the first and second flexible bands 255a, 255b also pass through the strain gauge 408b. During clamping, stapling, and cutting, a reaction force is exerted on anvil assembly 500 and the reload 400, which is communicated to support block 292, which then communicates the reaction force to a strain sensor of the strain gauge 408b.

Strain sensor of strain gauge 408b may be any device configured to measure strain (a dimensionless quantity) on an object that it is adhered to (e.g., support block 292), such that, as the object deforms, a metallic foil of the strain sensor is also deformed, causing an electrical resistance thereof to change, which change in resistance is then used to calculate loads experienced by trocar assembly 270. Strain gauge 408b provides closed-loop feedback to a firing/clamping load exhibited by first, second and third force/rotation transmitting/converting assemblies.

Strain sensor of strain gauge 408b then communicates signals to main controller circuit board 142. Graphics are then displayed on display 146 of the handle assembly 100 to provide the user with real-time information related to the status of the firing of handle assembly 100. Strain gauge 408b is also electrically connected to the electrical connector 312 (FIG. 3) via proximal and distal harness assemblies 314, 316.

For further details regarding the construction and operation of the circular stapler and its components, reference may be made to International Application Publication No. PCT/US2019/040440, filed on Jul. 3, 2019, the entire contents of which being incorporated by reference herein.

The reload 400 includes a storage device 402 and the circular adapter assembly 200 also includes a storage device 310 (FIG. 4). The storage devices 402 and 310 include non-volatile storage medium (e.g., EEPROM) that is configured to store any data pertaining to the reload 400 and the circular adapter assembly 200, respectively, including but not limited to, usage count, identification information, model number, serial number, staple size, stroke length, maximum actuation force, minimum actuation force, factory calibration data, and the like. In embodiments, the data may be encrypted and is only decryptable by devices (e.g., main controller 147) having appropriate keys. The data may also be used by the main controller 147 to authenticate the circular adapter assembly 200 and/or the reload 400. The storage devices 402 and 310 may be configured in read only or read/write modes, allowing the main controller 147 to read as well as write data onto the storage device 402 and 310.

Prior to operation of the powered circular stapler 10, the power handle 101 is enclosed within the shell housing 11 the adapter assembly 200 is coupled to handle assembly 100. After attachment of circular adapter assembly 200, handle assembly 100 initially verifies that circular adapter assembly 200 is coupled thereto by establishing communications with the storage devices 310 and 402 and authenticates the reload circular adapter assembly 200 and the reload 400. The data (e.g., usage count) stored on the storage devices 310 and 402 is encrypted and is authenticated by the power handle 101 prior to determining whether the usage count stored on the storage devices 310 and 402 exceeds the threshold (e.g., if the adapter assembly 200 has been previously used). Power handle 101 then performs verification checks (e.g., end of life checks, trocar member 274 missing, etc.) and calibrates circular adapter assembly 200 after the handle assembly 100 confirms that the trocar member 274 is attached.

The user commences a surgical procedure by positioning the adapter assembly 200, including the trocar member 274 and the anvil assembly 500, within the colorectal or upper gastrointestinal region. The user presses the toggle control button 30 to extend the trocar member 274 until it pierces tissue. After extension of the trocar member 274, the anvil assembly 500 that was previously positioned by surgeon is attached to the trocar member 274 and the user begins the clamping process on the tissue interposed between reload 400 and the anvil assembly 500 by pressing on the bottom portion of the toggle control button 30.

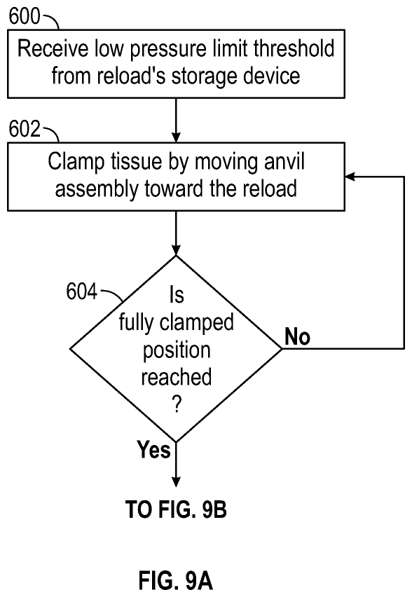

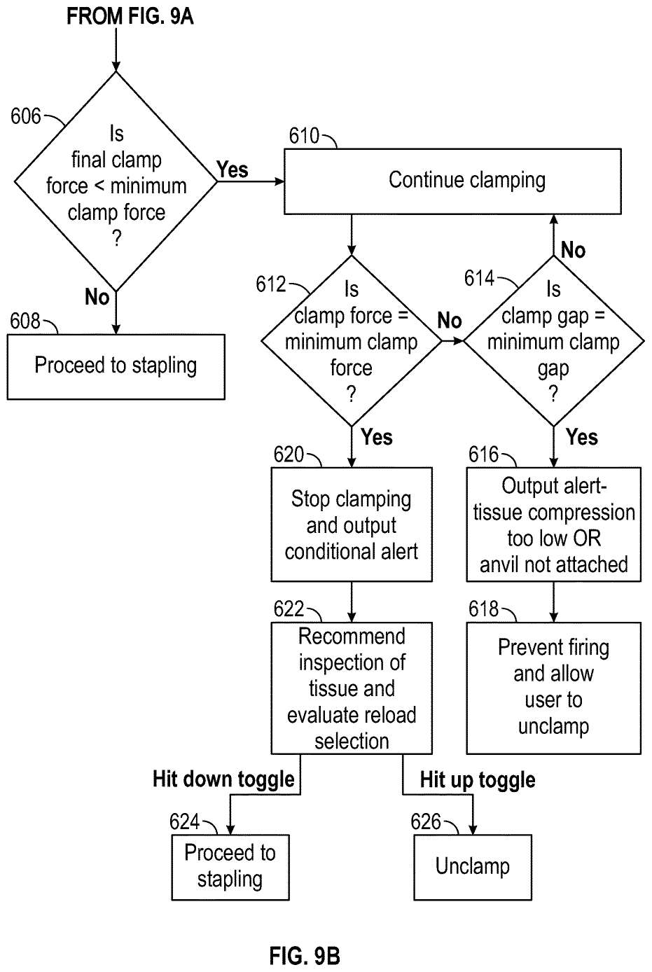

With reference to FIGS. 9A and 9B, the method for determining low tissue compression includes receiving at the power handle 101 from the storage device 141 or any other storage device of the stapler 10 data pertaining to the reload 400 at step 600. Data may include the size of the reload 400, denoting the diameter of a diameter from about 20 mm to about 35 mm. The diameter of the reload 400 is dependent on the size (diameter) of the colon, bowel, etc. being joined, which is determined by the surgeon. Data may also include clamp distance, which denotes a distance that the anvil 500 is moved to clamp tissue during the clamping phase. Clamp distance values may include first clamp distance, i.e., fully clamped distance, and a second clamp distance, i.e., minimal clamp distance that provides a larger clamp gap. In addition to the diameter, the data also may include the thickness information of reload 400, e.g., medium/thick (MT), extra thick (XT) as suited for different thickness tissue.

The data may also include various force limits, such as minimum and maximum clamp force limit, starting clamping force, target clamping force, etc. Starting clamping force may be any suitable threshold, e.g., from about 10 pounds to about 50 pounds. In embodiments, the target clamping force may be approximately 150 pounds. Minimum clamp force may be about 10 pounds and maximum clamp force may be about 250 pounds. In embodiments, the main controller 147 may calculate the minimum clamp force limit based on the size of the reload 400. More specifically, the main controller 147 may store a low tissue pressure limit and may calculate the minimum clamp force limit by multiplying the low tissue pressure limit by the surface area. The low tissue pressure limit may be derived empirically from anastomosis tissue testing.

After the anvil assembly 500 (placed in position by surgeon) is attached to the trocar member 274, at step 602, the clamping process is commenced on the tissue interposed between reload 400 and the anvil assembly 500 by the user pressing on the bottom of the toggle control button 30. During clamping, the anvil assembly 500 is retracted toward the reload 400 until reaching a preset, fully clamped position, namely a position of the anvil assembly 500 at which the tissue is fully clamped between the anvil assembly 500 and the reload 400. The position is continuously monitored at step 604. The preset, fully clamped position varies for each of the different types of reloads (e.g., the distance is about 0.61 mm for MT reloads, about 0.94 mm for XT reloads) and is based on the clamp distance read from the storage device 141. While clamping, the strain gauge 408b continuously provides measurements to the main controller 147 on the force imparted on the trocar member 274 as it moves the anvil assembly 500 to clamp tissue between the anvil assembly 500 and the reload 400. In addition, clamping time, i.e., time to reach the fully clamped position, and clamping pressure are also monitored as safety checks.

Once the anvil assembly 500 reaches the fully clamped position, at step 606, the main controller 147 compares the final clamp force, i.e., clamp force measured upon reaching the fully clamped position, to a minimum clamp force limit to determine whether sufficient tissue is disposed and compressed between the anvil assembly 500 and the reload 400. If the final clamp force is above the minimum clamp force limit, i.e., calculated by the main controller 147 or read from the storage device 141, this denotes that there is sufficient tissue present between the anvil assembly 500 and the reload 400 to proceed to stapling and cutting phases of the process in step 608.

If the final clamp force is below the minimum clamp force limit, this denotes that there is insufficient tissue present between the anvil assembly 500 and the reload 400. At step 610, the controller 147 continues the clamping process, during which the anvil assembly 500 continues to be retracted toward the reload 400 toward the minimum clamp gap. During this phase, continuously or at a predetermined sampling rate (e.g., 5 Hz), the controller 147 compares the clamp force to the minimum clamp force limit at step 612. If the measured clamp force is still not equal to (i.e., has not reached) the minimum clamp force, then at step 614, the controller 147 also determines whether the measured clamp gap (i.e., distance between the anvil assembly 500 and the reload 400) has reached the minimum clamp gap. If the minimum clamp distance has not been reached, then the controller 147 returns to step 610 to continue the clamping process.

Returning to step 614, if the clamp gap is equal to, i.e., has reached, the minimum clamp gap while the clamp force is not equal, i.e., has not reached, the minimum clamp force, then the controller 147 outputs a clamp alert at step 616. The alert may be a prompt shown on the display screen 146, which states that tissue compression is too low and/or that the anvil assembly 500 is detached.

At step 618, in addition to the clamp alert, the controller 147 disables subsequent phases, namely, ejecting staples and cutting. The only remaining action that is enabled is retraction, to allow the user to retry the process by unclamping and withdrawing the stapler 10.

Returning to step 612, if the measured clamp force is equal to the minimum clamp force, then at step 620, the controller 147 stops the clamping process and outputs another alert on the display screen 146 stating that the tissue pressure is too low, possibly due to insufficient tissue. The surgeon or assistant may verify the cause for the low pressure. At step 622, the controller 147 may also output another prompt recommending inspection of the tissue, evaluate selection of the reload 400, or recommend suturing. In particular if an XT reload 400 was used initially and the tissue pressure is detected as too low, then the MT reload 400 may be selected by the user and/or recommended by the controller 147.

Following the prompt, the user has an option to either proceed with the stapling at step 624, e.g., where no suitable alternative reload 400 is available, or to unclamp at step 626, e.g., to try another reload 400. Selection may be made via the toggle control button 30, e.g., by pressing down to proceed to stapling and cutting to step 624 and by pressing up to unclamp at step 626. At step 624, the controller 147 operates the stapler 10 in a “reduced clamp gap” condition and then automatically adjust the staple and cut strokes to compensate for the more proximal position of the anvil in order to ensure good staple formation and cutting of the anastomosis in this reduced gap state, which is less than normal clamp gap of the fully clamped position.

It will be understood that various modifications may be made to the embodiments of the presently disclosed adapter assemblies. Therefore, the above description should not be construed as limiting, but merely as exemplifications of embodiments. Those skilled in the art will envision other modifications within the scope and spirit of the present disclosure.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical device comprising:
a reload assembly including a plurality of staples;
an anvil assembly movable relative to the reload assembly;
a power source;
a motor coupled to the power source;
a transmission assembly movable by the motor and configured to move the anvil assembly relative to the reload assembly;
a force sensor configured to measure a force imparted on the anvil assembly by the transmission assembly;
a storage device storing data including a minimum clamp force limit; and
a controller configured to:
activate the motor to move the anvil assembly relative to the reload assembly;
compare the measured force to the minimum clamp force limit; and
output an alert in response to the measured force being lower than the minimum clamp force limit.

2. The surgical device according to claim 1, wherein the data further includes a clamping distance.

3. The surgical device according to claim 2, wherein the controller is further configured to activate the motor to move the anvil assembly relative to the reload assembly until the clamp distance is reached.

4. The surgical device according to claim 3, wherein the controller is further configured to compare the force measured at the clamping distance to the minimum clamp force limit.

5. The surgical device according to claim 1, wherein the controller is further configured to enable ejection of the plurality of staples in response to the measured force being higher than the minimum clamp force limit.

6. The surgical device according to claim 1, wherein the controller is further configured to determine an alternative reload assembly in response to the measured force being lower than the minimum clamp force limit.

7. The surgical device according to claim 6, further comprising:
a display screen configured to display at least one of the alert or information pertaining the alternative reload.

8. A method for controlling a surgical device, the method comprising:
receiving, at a controller, minimum clamp force limit stored in a storage device;
activating a motor to move a transmission assembly coupled to an anvil assembly that is movable relative to a reload assembly having a plurality of staples;
measuring, through a force sensor, force imparted on the anvil assembly by the transmission assembly;
comparing, at the controller, the measured force to the minimum clamp force limit; and
outputting, by the controller, an alert in response to the measured force being lower than the minimum clamp force limit.

9. The method according to claim 8, further comprising:
receiving, at the controller, a clamping distance stored in the storage device of the reload.

10. The method according to claim 9, further comprising:
activating the motor to move the anvil assembly relative to the reload assembly until the clamp distance is reached.

11. The method according to claim 10, further comprising:
comparing, at the controller, the force measured at the clamping distance to the minimum clamp force limit.

12. The method according to claim 8, further comprising:
enabling ejection of the plurality of staples in response to the measured force being higher than the minimum clamp force limit.

13. The method according to claim 9, further comprising:
determining, at the controller, an alternative reload assembly in response to the measured force being lower than the minimum clamp force limit.

14. The method according to claim 13, further comprising:
determining at the controller, in response to the measured force being less than the minimum clamp force at the clamping distance, to further retract the anvil until the minimum clamp force is achieved and then alerting of a low tissue compression condition and providing an option to proceed with ejection of the plurality of staples, in which case the controller reduces staple and cut stroke to compensate for more proximal location of the anvil.

15. A surgical device comprising:
a reload assembly including a plurality of staples and a storage device storing data pertaining to the reload assembly, the data including minimum clamp force limit and clamping distance;
an anvil assembly movable relative to the reload assembly;
a power source;
a motor coupled to the power source;
a transmission assembly movable by the motor and configured to move the anvil assembly relative to the reload assembly;
a force sensor configured to measure force imparted on the anvil assembly by the transmission assembly;
a display; and
a controller configured to:
activate the motor to move the anvil assembly relative to the reload assembly until the clamping distance is reached;
compare the force measured at the clamping distance to the minimum clamp force limit; and
output an alert on the display in response to the measured force being lower than the minimum clamp force limit.

16. The surgical device according to claim 15, wherein the controller is further configured to determine an alternative reload assembly thickness indication based on the measured force.

17. The surgical device according to claim 16, wherein the controller is further configured to output on the display, information pertaining the alternative reload assembly thickness indication.

18. The surgical device according to claim 15, wherein the controller is further configured to enable ejection of the plurality of staples in response to the measured force being higher than the minimum clamp force limit.
